(19) United States
(12) Patent Application Publication
(10) Pub. No.: US 2026/0151550 A1
(43) Pub. Date: Jun. 4, 2026

(54) DEVICE, SYSTEM AND METHOD FOR DETECTING BLOOD CLOTS

(71) Applicant: FRESENIUS MEDICAL CARE DEUTSCHLAND GMBH, Bad Homburg (DE)

(72) Inventors: Martin URBAN, Bad Homburg (DE); Manfred WEIS, Bad Homburg (DE); Ralf MÜLLER, Bad Homburg (DE); Christoph WIKTOR, Bad Homburg (DE); Arne PETERS, Bad Homburg (DE)

(73) Assignee: FRESENIUS MEDICAL CARE DEUTSCHLAND GMBH, Bad Homburg (DE)

(21) Appl. No.: 19/136,322

(22) PCT Filed: Dec. 7, 2023
(86) PCT No.: PCT/EP2023/084780

§ 371 Date: Jun. 6, 2025

(30) Foreign Application Priority Data
Dec. 12, 2022 (DE) 10 2022 133 061.3
Dec. 19, 2022 (DE) 10 2022 133 981.5

(51) Int. Cl. A61M 1/30; A61M 1/36

(52) U.S. Cl. CPC A61M 1/367; A61M 39/06; A61M 2039/0673; A61M 2205/3334

(57) ABSTRACT

To enable detection of blood clots in an extracorporeal blood circulation, a device for detecting blood clots in an extracorporeal blood circulation is proposed, the device optionally comprising an actuator which can create an adjustable constriction on the extracorporeal blood circulation in that it reduces the cross section of a section of a blood line, at least one sensor, which requires at least one parameter related to a property of the liquid in the extracorporeal blood circulation, and a control unit, which is configured such that a measurement signal from the at least one sensor is evaluated, and, that on the basis of this evaluation of the parameter measured by the sensor, the passage of a blood clot through the constriction and/or the sticking of a blood clot in the constriction can be detected.

The invention relates to the field of extracorporeal blood circulations.

The present invention relates to a device for detecting a blood clot in an extracorporeal blood circulation.

Extracorporeal blood circulations occur in the context of various therapeutic fields of use for medical treatments or also, for example, for blood plasma donation. Blood can experience environmental conditions in extracorporeal blood circulations which significantly differ from those within the endogenous blood circulation. Extravasal blood clots can form in extracorporeal blood circulations. Extravasal means that the clot is simply not located in an endogenous blood vessel. A blood clot within an endogenous blood vessel is called a thrombus. Blood clots are clumps of blood, thus consist of erythrocytes, leukocytes, and thrombocytes, which have formed because of beginning blood coagulation. From a certain size, blood clots can trigger negative health consequences when they reach the human blood circulation.

It is therefore an object of the present invention to provide a device, a system, and a method for detecting blood clots in an extracorporeal blood circulation.

### BRIEF DESCRIPTION OF THE INVENTION

The object is achieved by a device according to the invention for detecting blood clots in an extracorporeal blood circulation, connectable to an extracorporeal blood circulation having an adjustable constriction on a blood line, comprising:
at least one sensor, which acquires at least one parameter related to a property of the liquid in the extracorporeal circulation, and
a control unit, which is configured so that a measurement signal from the at least one sensor is evaluated, and that on the basis of this evaluation of the parameter measured by the sensor, the passage of a blood clot through the constriction and/or the sticking of a blood clot in the constriction can be detected,
wherein the device is configured so that it can detect blood clots at an adjustable constriction, which has been adjusted by constricting a blood tube using an actuator or a mechanical clamp.

One advantage of this device is that blood clots can be detected reliably in a noninvasive manner. The device according to the invention can detect not only the passage of a blood clot through the constriction, but also the partial clogging of the constriction by a blood clot which is sticking in the constriction.

The device according to the invention is provided and also configured for the purpose of being attached to an extracorporeal blood circulation or of existing as part of an extracorporeal blood circulation system. An extracorporeal blood circulation typically comprises blood lines or line sections, which are created, for example, using a tubing set. A blood line or a blood tube for an extracorporeal blood circulation is therefore generally elastic. According to the invention, the actuator of the device presented here can create an adjustable constriction on the extracorporeal blood circulation in that it reduces the cross-sectional area of a section of a blood line. The constriction of the cross section of a section of a blood line is preferably achieved by compressing the blood line, thus an elastic deformation. Accordingly, the deformation is reversible and when the actuator releases the blood line, it will again assume the shape which it had assumed before the creation of the constriction. Due to hygiene requirements, the blood lines, blood tubes, and tubing sets for extracorporeal blood circulations are generally only intended for single use and are disposed of after the first use. A blood treatment machine and other medical devices for use with an extracorporeal blood circulation, in contrast, are generally used for hundreds or thousands of treatments. For a blood treatment using an extracorporeal blood circulation, a combination of a repeatedly usable treatment machine and a blood tube provided only for single use are thus typically used in a combination. The device presented here for detecting blood clots is intended for repeated use and can be part of a treatment machine. However, it can also be designed as a device separate from a treatment machine.

In the context of the present application, a constriction means a line section on which the line cross section is smaller than on the remaining line. The device presented here or a system building thereon and the method presented here relate to an adjustable constriction which can be created or is created on an extracorporeal blood circulation. In general, an adjustable constriction means both, on the one hand, that the constriction can so to speak be “added on”, i.e. can be formed by actuating an actuator. On the other hand, however, an adjustable constriction also means that the width-thus the cross section remaining after the constriction—can in general assume different values. This applies for the various embodiments. The invention relates to the detection of a passage of a blood clot through a constriction in an extracorporeal blood circulation. That is to say, the constriction can be of a nature such that the blood circulation through the adjustable constriction is not interrupted. The adjustable constriction in the meaning of the invention can thus be a line section which has a smaller cross section than the surrounding line, but the line is not completely clamped off. The blood line cannot be sealed or closed at the adjustable constriction in the meaning of the invention when the adjustable constriction is adjusted. Rather, a liquid can flow through the adjustable constriction in the meaning of the invention. A line section of a blood tube set which is completely clamped off by a closed safety clamp is, for example, completely sealed/impermeable for a liquid in an extracorporeal blood circulation. It becomes clear from this that the last-mentioned example clearly is not an adjustable constriction in the meaning of the invention. A valve in the extracorporeal blood circulation which is completely closed is also not an adjustable constriction in the context of the invention. A mechanical means which forms an adjustable constriction on a section of a blood line in the meaning of the invention can so to speak be located in a partially open position with regard to the cross section of the section of the blood line in the mechanical means. Partially open means that the line section is reduced by the mechanical means in relation to a section without constriction, but at the same time is not completely squeezed off, so that a liquid can still flow through the constriction. Mechanical means can in this case be an actuator, e.g., an electrically controlled linear actuator, or an electric or electromagnetic safety clamp, or else a tube clamp to be actuated manually, wherein all of these means reduce a tube cross section by squeezing closed in the context of the invention, so that a half-open position is implemented.

The tube clamp to be actuated manually can also be sold as a disposable part for single use integrated in an article such as a blood tube set.

The detection of blood clots in the context of the invention can be used to increase the level of patient safety. In optional refinements, a protective measure can be initiated as a result of the detection of a blood clot. Such protective measures can be, for example, closing a safety tube clamp, stopping a blood pump, interrupting the conveyance in the extracorporeal blood circulation in general, or also outputting an alarm or notifying patients and/or medical treatment personnel.

With respect to the invention, in general when reference is made to the detection of the passage of a blood clot through a constriction, the detection of sticking of a blood clot in this constriction is alternatively or additionally meant.

Furthermore, the object is achieved by a method according to the invention for monitoring an extracorporeal blood circulation for detecting a blood clot in the extracorporeal blood circulation having at least the steps:
producing a constriction on the blood circulation, on a section of a blood tube, using an actuator or using a mechanical means for manually producing a constriction,
measuring at least one parameter related to a property of the liquid in the line section having the constriction using a sensor,
evaluating the measurement data of the sensor to detect the passage of a blood clot through the constriction and/or the sticking of a blood clot in the constriction using a control unit.

“Creating a constriction” on the blood circulation can be understood as synonymous with “producing a constriction”. The constriction is created using an actuator. The blood circulation is often formed using an elastic blood tube. The constriction can then be understood as a temporary constriction, because it is created by the actuator and can also be eliminated again by the actuator and by the reset of the blood tube. One advantage of this method is that blood clots can be detected reliably in a noninvasive manner.

In the context of the present disclosure, the production of a constriction on the blood circulation primarily means the production of a constriction on a section of a blood tube which forms at least a part of the extracorporeal blood circulation, using an actuator or using a mechanical means for manually producing a constriction, for example, in that the cross section of the blood tube is less in the constriction than on the blood tube outside the constriction.

In order to serve the above-mentioned purpose of increasing the level of patient safety, the detection of blood clots on the extracorporeal blood circulation can preferably take place at a distance to the patient access such that a safety measure can be taken. A constriction directly at the location of the return of the patient blood, thus a cannula, would therefore be disadvantageous to serve for this purpose: If a blood clot is detected during the passage through the cannula from the extracorporeal blood circulation into the patient blood circulation, for example, it is not possible to prevent the blood clot from reaching the blood circulation of the patient by stopping the conveyance in the extracorporeal blood circulation. This can be because of the inertia of the system, because of which a time delay of the blood flow stop and/or the alarm processing can occur, due to which, for example, a clot could therefore still reach the patient in spite of this detection. However, a cannula is also not an adjustable constriction in the context of the present application for other reasons. On the one hand, the constriction is not adjustable, but rather fixed—as part of the extracorporeal blood circulation. The constriction of a cannula cannot be created/generated in contrast to the context of the present application. On the other hand, the width of the cannula is not necessarily suitable to ensure reliable detection of clots. The sensitivity of the detection of clots is dependent on the width of the constriction and further factors such as flow, pressure, and viscosity also play a role.

The device according to the invention for detecting blood clots is configured not only to detect the passage of a blood clot, but also the partial or complete clogging of the constriction by a blood clot. The partial or complete clogging of the constriction by a blood clot is also possible using the method according to the invention. Sticking can also be referred to instead of clogging. The detection of the passage of a blood clot is in the foreground of this application.

The present invention relates to a device for detecting blood clots in an extracorporeal blood circulation. The device has a control unit which is configured to detect a blood clot at an adjustable constriction in a blood circulation in that a sensor which acquires at least one parameter related to a property of the liquid in the extracorporeal circulation. The control unit is configured here so that a measurement signal from the at least one sensor is evaluated, and that on the basis of this evaluation of the parameter measured by the sensor, the passage of a blood clot through the constriction can be detected.

The present invention furthermore relates to a method for monitoring an extracorporeal blood circulation for detecting a blood clot in the extracorporeal blood circulation having at least the steps:
producing a constriction on the blood circulation using an actuator or using a mechanical means for manually producing a constriction,
measuring at least one parameter related to a property of the liquid in the line section having the constriction using a sensor,
evaluating the measurement data of the sensor to detect the passage of a blood clot through the constriction or the sticking of a blood clot in the constriction.

This means here that an adjustable constriction is formed and is maintained for at least the period of time of the measurement. This can be viewed as an implicit step.

For methods and devices, it holds true that the measurement for detecting a clot at the constriction takes place over a minimal predetermined period of time in which the adjustable constriction is not varied. The width is thus maintained for this period of time. In certain embodiments of the inventive methods and devices, the adjustable constriction is adjusted and maintained after it is produced at least for a predetermined period of time of, for example, a second, so that a measurement can take place. Other periods of time down to less than a few tens of milliseconds or up to two-digit numbers of seconds or also for several minutes continuously, for example, 15 minutes, are conceivable. The short measurements can advantageously be executed intermittently to interfere with the progress less. The long measurements are advantageous for certain operating states or modes of a blood treatment, so that if necessary an entire operating phase such as closed-loop reinfusion can be completely monitored. It also becomes clear from this that the adjustable constriction does not mean an infinitesimal intermediate state which occurs when, for example, a tube clamp closes in case of emergency and different widths exist for fractions of a second there during the closing. This unplanned coincidence is not meant.

The device according to the invention can have an actuator for the production or can be embodied without an actuator. In the case in which it is embodied without the actuator, it is configured to detect blood clots at a manually produced constriction in operation. This constriction can be induced, for example, by applying a mechanical means for producing a constriction on a blood tube or on a blood line. In a central refinement, the control unit is moreover configured, upon input of a user, to change to the operating mode for detecting blood clots or to change to an operating mode without the detection of blood clots or alternatively to detect on the basis of other parameters of an extracorporeal blood treatment when a mode for detecting blood clots is to be activated or deactivated, and to change independently between an operating mode with and without detection of blood clots. This detection can be carried out, for example, on the basis of the detection of the reverse running of a blood pump of a blood treatment device or on the basis of the detection of an elevated pressure in the blood tube, which was induced by the setting of the constriction. Both detections (running direction of a blood pump, assuming a higher base pressure in the blood tube) are combinable. The device according to the invention can be parts of a blood treatment device or a separate device connectable to a blood treatment device. Furthermore, a system according to the invention is disclosed which comprises a device for detecting blood clots, a blood tube or a blood line or a blood tube set, which are configured for connection to a patient and/or a blood treatment device. One refinement of the system furthermore comprises, in addition to the device for detection and the blood tube, a mechanical means for producing a constriction on a blood circulation. According to one aspect of the invention, a system is disclosed for use with a device or a method for detecting a blood clot at a constriction in an extracorporeal blood circulation comprising a blood tube or a blood line or a blood tube set configured for producing an extracorporeal blood circulation and a mechanical means for producing an adjustable constriction on a line section of the blood tube or the blood line or the blood tube set. In one embodiment of the invention, a system made up of a blood treatment machine and a device for detecting blood clots and a blood tube or a blood line or a blood tube set and a mechanical means for producing an adjusting constriction on a line section is formed. The blood tube set on the one hand and the device for detecting blood clots on the other hand, which can be part of a blood treatment machine or separate therefrom, together form-like plug and socket—in some embodiments, for example, the pair made up of consumable product and evaluation device for detecting blood clots, which can optionally be carried out using a method according to the invention, for example, because device, blood tube set, and systems comprising device and/or blood tube set and method are all aspects of this invention. Several refinements and embodiments of aspects of the invention, of methods and devices and systems are discussed hereinafter. Provided they are not technically contradictory, refinements, embodiments, and aspects described hereinafter always mean both device and method and concepts comprising them as well as the system.

In one refinement of the device according to the invention for detecting blood clots in an extracorporeal blood circulation, the sensor is a flow sensor that measures the current flow rate of the liquid or a pressure sensor that measures the current pressure of the liquid. These two sensor types particularly advantageously enable a robust detection of blood clots.

A further refinement of the device according to the invention for detecting blood clots in an extracorporeal blood circulation has at least both one flow sensor and at least one pressure sensor. Furthermore, in this refinement, the control unit of the device is configured such that it uses the measured values of pressure sensor and flow sensor in consideration together to detect blood clots. Due to the joint evaluation of measured values of pressure and flow, the sensitivity of the detection of blood clots may particularly advantageously be increased.

One embodiment of the device according to the invention for detecting blood clots in an extracorporeal blood circulation has an actuator, which can create an adjustable constriction on the extracorporeal blood circulation in that it reduces the cross section of a section of a blood line. The possibility is thus particularly advantageously provided that the device creates the constriction required for detection without action of a user.

One refinement of the device according to the invention for detecting blood clots in an extracorporeal blood circulation having an actuator is designed as a tube clamp or as part of a tube clamp into which a medical tube is insertable. Tube clamps are widespread in many medical devices, for example, to prevent as a safety system a blood loss of the patient in the event of a disturbance (for example power failure) or a handling error or accident (for example unnoticed slipping out of a cannula). Such tube clamps are typically designed so that a blood tube can be inserted into a defined receptacle and the clamp is held open in normal operation. In the event of power failure, the clamp closes in a fraction of a second, for example, due to spring pretension or magnetic pretension. The embodiment of the device according to the invention as a tube clamp or part of a tube clamp provides as advantages increased cost-effectiveness, because elements of a clamp provided in any case are also used in the context of the invention. On the other hand, the solution is also very compact. Moreover, safety aspects may be increased.

In one refinement of the device according to the invention for detecting blood clots in an extracorporeal blood circulation, which is designed as a tube clamp or as part of a tube clamp into which a medical tube is insertable, the actuator can assume at least three different positions. These positions are: a closed position, in which the extracorporeal blood circulation does not have a free line cross section at a point, i.e., is blocked or the tube is clamped off by the clamp; an open position, in which the extracorporeal blood circulation does not have a constriction at the actuator, and a third, partially open position, which is between the closed and the open position, in which an adjusted constriction can be created to detect blood clots on an inserted tube. In the first position, the tube is thus not permeable, rather is completely pressed together/clamped off/squeezed off. The effective cross section for the liquid transport in the constriction is zero in this case. In the second position, the tube is not impaired by the clamp in the cross section, thus is maximal. In the third position, a defined constriction is formed on the tube in that the clamp is half-open. This defined constriction is designed for the inventive detection of blood clots. The particular advantage of a clamp having three positions is that it can thus likewise fulfill the function of a typical tube clamp, but in the half-open position can form a device according to the invention for detecting blood clots.

In one refinement of the device according to the invention for detecting blood clots in an extracorporeal blood circulation, wherein the actuator is designed as a tube clamp or as part of a tube clamp into which a medical tube is insertable, the actuator can assume at least three different positions. These positions are: a closed position, in which the extracorporeal blood circulation does not have a free line cross section at a point, i.e., is blocked or the tube is clamped off by the clamp; an open position, in which the extracorporeal blood circulation does not have a constriction at the actuator, and a third, partially open position, which is between the closed and the open position, in which an adjusted constriction can be created to detect blood clots on an inserted tube. In the first position, the tube is thus not permeable, rather is completely pressed together/clamped off/squeezed off. The effective cross section for the liquid transport in the constriction is zero in this case. In the second position, the tube is not impaired by the clamp in the cross section, thus is maximal. In the third position, a defined constriction is formed on the tube in that the clamp is half-open. This defined constriction is designed for the inventive detection of blood clots. The particular advantage of a clamp having three positions is that it can thus likewise fulfill the function of a typical tube clamp, but in the half-open position can form a device according to the invention for detecting blood clots.

In one refinement of the device according to the invention for detecting blood clots in an extracorporeal blood circulation, which is designed as a tube clamp or as part of a tube clamp into which a medical tube is insertable, and wherein the actuator can assume at least three different positions, the tube clamp has so-called polymagnets. Polymagnets have a large number of magnetized areas so that the clamp has at least three different positions. Polymagnets are disk-shaped magnets made of a ferromagnetic material which can be variably magnetized in small parts perpendicular to the disk face by applying an external magnetic field. Various clamp positions may be adapted particularly advantageously for the application according to the invention by the use of such very finely adaptable magnets.

In one refinement of the device according to the invention for detecting blood clots in an extracorporeal blood circulation, wherein the actuator is designed as a tube clamp or as part of a tube clamp into which a medical tube is insertable, and wherein the actuator can assume at least three different positions, the tube clamp has so-called polymagnets. Polymagnets have a large number of magnetized areas so that the clamp has at least three different positions. Polymagnets are disk-shaped magnets made of a ferromagnetic material which can be variably magnetized in small parts perpendicular to the disk face by applying an external magnetic field. Various clamp positions may be adapted particularly advantageously for the application according to the invention by the use of such very finely adaptable magnets.

In one refinement of the device according to the invention for detecting blood clots in an extracorporeal blood circulation, which is designed as a tube clamp or as part of a tube clamp, into which a medical tube is insertable, a spacer can be arranged between clamp elements of the clamp in order to thus implement the partially open clamp position at the constriction. It could be thought that this arrangement would contradict the actual intended use of a tube clamp as a safety device. However, on the one hand, it is conceivable to design a tube clamp so that the clamp can still close completely and only partial closing of the clamp is already caused by the spacer. On the other hand, it is conceivable to refit a tube clamp for detecting blood clots by using the specific spacer and configuration of the control unit to detect blood clots on the basis of sensor data. A device according to the invention may thus be produced particularly advantageously with reduced expenditure by modification of existing machine elements and the development time may be shortened. The high degree of identical parts with an already existing technology is also advantageous. A clamp refitted to create a constriction for detecting blood clots cannot be readily used as a conventional safety clamp. In some applications, such as hemodialysis, a clamp refitted in this way would then be able to be viewed in addition to the clamps required with respect to safety. The device embodied according to this aspect only optionally has an actuator for producing the constriction. Variants do not have an actuator: A constriction is then implementable solely by means of spacers. Other variants have an actuator, so that a constriction can alternately be implemented using the actuator or using the spacer. It is in turn possible here that a different width of the constriction is implementable using the spacer than using the actuator. The constriction can thus advantageously be adapted in a cost-effective manner to individual parameters of a blood treatment. It is conceivable that a refitted clamp comprises a spacer which is arranged so it can be folded or moved in another manner on the clamp, so that if needed it can be inserted into the clamp in such a way that a constriction is formed. On the other hand, it is conceivable to provide a clamp and a separate spacer as a kit. As a refinement thereof, it is conceivable to provide a kit having multiple different or also identical spacers.

In one refinement of the device according to the invention for detecting blood clots in an extracorporeal blood circulation, wherein the actuator is designed as a tube clamp or as part of a tube clamp into which a medical tube is insertable, a spacer can be arranged between clamp elements of the clamp to thus implement the partially open clamp position at the constriction. It could be thought that this arrangement would contradict the actual intended use of a tube clamp as a safety device. However, on the one hand, it is conceivable to design a tube clamp so that the clamp can still close completely and only partial closing of the clamp is already caused by the spacer. On the other hand, it is conceivable to refit a tube clamp for detecting blood clots by using the specific spacer and configuration of the control unit to detect blood clots on the basis of sensor data. A device according to the invention may thus be produced particularly advantageously with reduced expenditure by modification of existing machine elements and the development time may be shortened. The high degree of identical parts with an already existing technology is also advantageous. A clamp refitted to create a constriction for detecting blood clots cannot be readily used as a conventional safety clamp. In some applications, such as hemodialysis, a clamp refitted in this way would then be able to be viewed in addition to the clamps required with respect to safety. The device embodied according to this aspect only optionally has an actuator for producing the constriction. Variants do not have an actuator: A constriction is then implementable solely by means of spacers. Other variants have an actuator, so that a constriction can alternately be implemented using the actuator or using the spacer. It is in turn possible here that a different width of the constriction is implementable using the spacer than using the actuator. The constriction can thus advantageously be adapted in a cost-effective manner to individual parameters of a blood treatment. It is conceivable that a refitted clamp comprises a spacer which is arranged so it can be folded or moved in another manner on the clamp, so that if needed it can be inserted into the clamp in such a way that a constriction is formed. On the other hand, it is conceivable to provide a clamp and a separate spacer as a kit. As a refinement thereof, it is conceivable to provide a kit having multiple different or also identical spacers.

In one refinement of the device according to the invention for detecting blood clots in an extracorporeal blood circulation, the actuator and the control unit are configured so that the device can assume at least two different operating states in each of which the actuator forms the constriction, wherein the predetermined width of the constriction is different in the different operating states. The device thus has at least two different predetermined operating states of predetermined width at the constriction here, wherein the predetermined width is of a nature such that the blood flow through the constriction does not completely come to a stop, thus it is not a completely occluding constriction. This makes it possible to select the width of the constriction so that a parameter of the extracorporeal blood circulation is taken into consideration to obtain the highest available sensitivity or reliability of the detection of blood clots. A parameter can be the volume flow rate of the blood. In one embodiment, for example, a lesser width of the constriction is selected if a lower volume flow rate is present. A parameter can be the diameter or the wall thickness of the blood line/the blood tube. A parameter can be the viscosity or the hematocrit of the blood or the progress over time of the treatment. A parameter can be a set delivery rate of a blood pump. A further parameter can be the pressure in the blood tube when the constriction is still open. A parameter can be a value which is formed from the pressure in the tube and the volume flow rate in the tube. In addition to the parameter of pressure, a pressure increase upon the creation of the adjustable constriction with known blood volume flow can be a further parameter. In this way, for example, the influence of a patient connector such as a cannula on the pressure measurement can advantageously be compensated for.

In the two different predetermined operating states having different predetermined width at the constriction, in both cases the blood tube is not completely clamped off in the area of the adjustable constriction here, but rather the blood in the extracorporeal blood circulation can flow through the constriction.

In one refinement of the device according to the invention for detecting blood clots in an extracorporeal blood circulation, which is designed as a tube clamp or as part of a tube clamp into which a medical tube is insertable, and in which the actuator can assume at least three different positions, the actuator can assume at least one further partially open fourth position for setting predetermined constrictions, wherein the predetermined width of the constriction is different in the different partially open positions. The advantages of the refinement described in the preceding paragraph also apply in this case.

All aspects and embodiments of the present invention share the feature that an adjustable constriction for detecting blood clots is formed, wherein an actuator or a mechanical means assumes a partially open position. Partially open also relates to the cross section of the section of the blood tube which is located at the location of the adjustable constriction: The free cross section of the blood tube is reduced here in relation to the relaxed blood tube, as could be present on sections before and/or after the adjustable constriction. The free cross section is only reduced enough here so that a flow of a liquid in the tube section through the constriction remains possible.

In one refinement of the device according to the invention for detecting blood clots in an extracorporeal blood circulation, the actuator and the control unit are configured so that the device can assume at least two different operating states, in each of which the actuator forms the constriction, wherein the predetermined width of the constriction is different in the different operating states, and the control unit is configured, for detecting blood clots in consideration of a parameter of the extracorporeal blood circulation, to select the width of the constriction according to a predetermined scheme and set it on the actuator. Automation of the device is advantageously possible by way of this additional configuration of the control unit to set the width of the constriction in consideration of a parameter. Furthermore, the detection capability of blood clots is advantageously improved if the setting best suitable for detection can thus be selected in each case by the device.

In one refinement of the device according to the invention for detecting blood clots in an extracorporeal blood circulation, which is designed as a tube clamp or as part of a tube clamp into which a medical tube is insertable, and in which the actuator can assume at least three different positions and at least one further partially open fourth position for setting predetermined constrictions, wherein the predetermined width of the constriction is different in the different partially open positions, the control unit is configured, for the operation for detecting blood clots in consideration of a parameter of the extracorporeal blood circulation, to select the clamp position according to a predetermined scheme and set it on the actuator. The advantages described in the preceding paragraph relating to the exemplary embodiment there also apply in this case.

In one refinement of the device according to the invention for detecting blood clots in an extracorporeal blood circulation, the width of the constriction is variable and the control unit is configured to set the constriction for the operation for detecting blood clots in consideration of a parameter of the extracorporeal blood circulation so that the sensitivity and/or the robustness of the detection is optimized. One advantage of this refinement is that the sensitivity or the robustness of the detection can be even better than for embodiments in which one or more predetermined widths of the constriction are specified. It is conceivable, for example, that a parameter of the extracorporeal blood circulation is in the middle between two values which require a specific width of the constriction according to the scheme for optimum results. An individual width of the constriction can be assumed here using the variable constriction of this embodiment and the detection can thus be improved in relation to a set of fixed widths. In a further embodiment, the control unit is configured to select a respectively optimized width of the constriction variably for specific previously known blood tubes or blood tube sets. Alternatively or additionally, the control unit can be configured to adapt the width of the constriction to various tubing sets or blood tubes or blood lines or blood tube sets. One advantage would be that blood clots are detected easily in the same device even with various blood tubes—for example, for the treatment of adults or else designed for the treatment of children, who have significantly thinner tubes (smaller internal diameter and/or lower wall thickness of the blood tube/the blood line). For this purpose, for example, the control unit can be configured to process a parameter of the blood tubes such as wall thickness or internal diameter as an input value for the adapted setting of the constriction. Depending on the embodiment, the control unit can be configured to have such a value entered by hand, for example, or to have it transmitted from another device or other components of a device, for example, if the device is part of an extracorporeal blood treatment machine.

In one refinement of the device according to the invention for detecting blood clots in an extracorporeal blood circulation, a roller pump is the actuator and the constriction is created in that a movable mechanical means for creating the constriction is arranged at or adjacent to the roller pump so that in case of reverse running of the pump, the mechanical means moves and thus creates the constriction.

In one refinement of the device according to the invention for detecting blood clots in an extracorporeal blood circulation, the width of the constriction is variable and the control unit is configured to adjust the constriction for the operation for detecting blood clots in consideration of a parameter of the extracorporeal blood circulation so that the sensitivity and/or the robustness of the detection is optimized and the control unit is configured to calculate the optimum width of the constriction and set it using the actuator during a medical treatment using the extracorporeal blood circulation as a function of a parameter of the extracorporeal blood circulation and/or a parameter of the treatment. Automated, individually improved detection of blood clots is advantageously enabled in this way. Human errors are reduced and the capacities of medical personnel are strained less by the automation of the device.

One embodiment of the invention relates to a blood tube set, configured for use with a device presented here for detecting blood clots in an extracorporeal blood circulation or with a method presented here for detecting blood clots in an extracorporeal blood circulation, the blood tube set having a blood tube or a blood line and a mechanical means, in particular a mechanical clamp, for adjusting a constriction on a line section of the blood tube or the blood line. The blood tube set suitable for detecting blood clots is advantageously provided to the user in this way.

One aspect of the present invention relates to an extracorporeal blood treatment device having a device according to the invention for detecting a blood clot, a tube clamp, and/or a means for outputting an alarm and/or a blood pump, wherein the control unit of the blood treatment device is configured to close the tube clamp and/or output an alarm and/or stop the blood pump if the device for detecting blood clots detects a blood clot. In other words, according to this aspect, an extracorporeal blood treatment device according to the invention is equipped with a device according to the invention for detecting a blood clot. In addition, the extracorporeal blood treatment device has one or more of the active components of this list: tube clamp, means for outputting an alarm, blood pump. The control unit is configured here so that upon detection of a blood clot, at least one activation of a component takes place in reaction to the detection of a blood clot. This particularly advantageously enables a control-technology response to the detection of a blood clot to be obtained directly. Different variants thereof are comprised. For example, one variant of the invention is comprised in which the extracorporeal blood treatment device has a device according to the invention for detecting blood clots and a tube clamp, wherein the control unit is configured to close the tube clamp when the device detects a blood clot. Alternatively or additionally, for example, a variant of the invention is comprised in which the extracorporeal blood treatment device has a device according to the invention for detecting blood clots and a means for outputting an alarm, wherein the control unit is configured to output an alarm when the device detects a blood clot. Alternatively or additionally, for example, a variant of the invention is comprised in which the extracorporeal blood treatment device has a device according to the invention for detecting blood clots and a blood pump, wherein the control device is configured to stop the blood pump when the device detects a blood clot. These three variants can moreover be combined as desired. Thus, for example, in one variant having tube clamp and blood pump, the control device can be configured so that upon detection of a blood clot, both the tube clamp is closed and the blood pump is stopped. Furthermore, for example, in one variant having blood pump and means for outputting an alarm, the control unit can be configured so that upon detection of a blood clot, the blood pump is stopped and moreover an alarm is output. In addition, in one variant having means for outputting an alarm and tube clamp, the control unit can be configured so that upon detection of a blood clot, an alarm is output and in addition the tube clamp is closed. Or, for example, in one variant that has means for outputting an alarm, a tube clamp, and a blood pump, the control unit can be configured to output an alarm upon detecting a blood clot, to close the tube clamp, and to stop the blood pump. Further variants are conceivable. Variants having multiple active means, i.e., multiple blood pumps, multiple tube pumps, or multiple means for outputting an alarm are also thus conceivable. The control unit can then be configured here to carry out the above-mentioned actions in one or more of the active means upon detection of a blood clot. One or more blood pumps can thus be stopped, one or more tube clamps can be closed, or an alarm can also be output on one or more means for outputting an alarm. It is moreover conceivable to output multiple alarms by means of one means for outputting an alarm.

According to one aspect of the invention, the above-mentioned extracorporeal blood treatment device having a device according to the invention for detecting a blood clot is a hemodialysis machine. The device according to the invention for detecting blood clots may advantageously be used in cooperation with a hemodialysis machine or as a component thereof. Particular synergies result therefrom, because some elements of a typical hemodialysis machine may also be used as elements of the device for detecting blood clots or vice versa, elements of the device can also expand the hemodialysis machine beyond the detection of blood clots. A pressure sensor or blood volume flow sensor can be such an element, for example. In addition, the invention is also a particular advantage for the context of hemodialysis because there is an existing interest in detecting blood clots.

According to one aspect of the invention, a system for extracorporeal blood treatment of a patient has an extracorporeal blood treatment machine and the extracorporeal blood treatment machine has a device according to the invention for detecting a blood clot in an extracorporeal blood circulation. The system additionally has a blood tube. The device for detecting blood clots is arranged on a section of the blood line which is at a distance of at least 30 cm upstream of the access of the patient. This advantageously enables blood clots to be detected by the device at a significant distance from the patient. This distance corresponds somewhat more than to what is covered at a blood volume flow of 200 ml/min at an internal diameter of the tube of 4.5 mm in a period of time of 1.5 s. This time span is sufficient for the reaction of a safety tube clamp. In one embodiment, the section of the blood line having the constriction is arranged as a function of the blood volume flow rate provided for an individual treatment and in consideration of the internal diameter of the blood tube to be used so that blood in measurement operation for detecting a blood clot would reach the patient at earliest 1.5 s after passing the constriction. This embodiment is primarily an alternative to the embodiment, wherein the distance is at least 30 cm. Since in this embodiment the distance is calculated using a formula, however, it can also be that the formula results in a distance of 30 cm or more and therefore the two embodiments have an overlapping parameter range. The distance x (Q, D, T) in [cm] as a function of blood volume flow rate Q in [ml/s=cm³/s], tube internal diameter D in [cm] and the desired period of time T in [s] between detection of the clot and standstill of the blood conveyance in the extracorporeal blood circulation, can be calculated as follows:

$$x ( Q , D , T ) = \frac{Q [ \frac{{cm}^{3}}{s} ] * T [ s ]}{π * {( \frac{D}{2} )}^{2} [ {cm}^{2} ]}$$

Here, with Q=200 cm³/s; D=4.5 mm; T=1.5 s, a value of approximately 18.9 cm is obtained for the distance x. At the above-specified 30 cm, the distance is thus in any case greater than the blood volume would transport from the constriction under these conditions within 1.5 seconds. An arrangement of the adjustable constriction at a distance to the patient access according to the above-mentioned formula is conceivable for all aspects of the invention.

According to a further aspect of the invention, the distance of the adjustable constriction to the patient access can also alternatively be determined according to the following formula, however. The device for detecting blood clots is arranged here on a section of the blood line which is determined as follows as a function of the blood volume flow rate and the tube diameter:

$$x = 30 cm * \frac{Q}{200 [ \frac{ml}{min} ]} * \frac{4.5 [ mm ]}{D}$$

In this case, x is the distance in cm, Q is the blood volume flow rate in [ml/min], D in [mm] is the free internal diameter of the conventional general diameter of the blood tube line, where no adjustable constriction is formed. This advantageously enables a minimal value determined by parameters of the treatment to be selected for the distance of the adjustable constriction, wherein safe stopping of the blood conveyance is still enabled so that a clot detected in the adjustable constriction remains in the extracorporeal circulation. At a conveyance rate of 300 ml/min and an internal diameter of the relaxed blood tube, a distance of 45 cm thus results. An arrangement of the adjustable constriction at a distance to the patient access according to the above-mentioned formula is conceivable for all aspects of the invention.

An embodiment of a method presented here therefore additionally comprises the step of determining the above-mentioned distance. The step is placed upstream of the creation of the adjustable constriction. The spatial distance moreover advantageously enables a longer reaction time or time for detecting a blood clot and processing the detection. As a refinement, a greater safety distance is conceivable, for example, corresponding to 2 s or 3 s or a fixed distance such as 50 cm or 100 cm.

In one refinement of the method according to the invention, the method for monitoring an extracorporeal blood circulation for detecting a blood clot is expanded such that it also comprises an aspect of the control of the extracorporeal blood treatment device. The method for controlling an extracorporeal blood treatment device has, in addition to the steps of detecting a blood clot, the step here: Closing a tube clamp and/or outputting an alarm and/or stopping a blood pump when the passage of a blood clot through the constriction is established. The above-mentioned aspects of the device according to the invention which comprise closing a tube clamp and/or outputting an alarm and/or stopping a blood pump when the passage of a blood clot through the constriction or the sticking of a blood clot in the constriction is established also apply to subforms of the method.

One purpose of the alternatives of the above-mentioned refinement which are directed to closing a tube clamp and/or stopping a blood pump is that upon detection of a blood clot at the adjustable constriction, the clot does not reach the patient. It is then an implied condition that the adjustable constriction is arranged at a distance from both patient-side ends of the blood tube so that upon detection of the passage of a clot through the adjustable constriction, there is still sufficient reaction time so that upon closing of the tube clamp and/or stopping of the blood pump, the blood volume having the detected clot does not reach the patient. I.e., the distance—along a blood tube—has to be selected at least so that there is sufficient time for the control of the extracorporeal blood circulation and the reaction of the actuators—also in consideration of the inertia of the respective system—in order to prevent the blood transport of the blood volume having the detected clot to the patient.

The terms identification of a blood clot and detection of a blood clot can be used synonymously.

According to one aspect, the present device for detecting blood clots in an extracorporeal blood circulation is connected to an extracorporeal blood treatment machine or part of an extracorporeal blood treatment device, wherein the extracorporeal blood treatment device is configured, upon ending of the extracorporeal blood treatment, to infuse the blood located in the extracorporeal blood circulation back to the patient by means of closed-loop reinfusion. This particularly advantageously enables the level of safety of the reinfusion, the blood treatment, and the extracorporeal blood treatment device to be increased. For example, the extracorporeal blood treatment machine in this example can be a hemodialysis machine or a machine for therapeutic apheresis.

According to one aspect, the present device for detecting blood clots in an extracorporeal blood circulation is designed as a safety system for extracorporeal blood circulations so that the device can be added later to an already existing extracorporeal blood treatment device or an existing extracorporeal blood circulation (so-called retrofitting). The device is embodied as a retrofit kit here so that, for example—as required—it can establish a wired or wireless data connection with a blood treatment machine or manages without a data connection, that it has an independent electrical energy supply (for example by means of a potential-free isolation transformer from the power grid or by means of an accumulator), or is powered by a blood treatment machine, that it can simply be clamped using a clip on a blood tube. In a completely independent variant, the device optionally has a separate display means, a separate means for outputting an alarm, and/or a transmission device for transmitting measured values and/or alarm signals. The methods according to the invention are also to be viewed as transferred to this aspect.

According to one aspect of the present invention, the control unit is configured to detect whether the actuator is in an open state or whether the actuator forms a constriction on the extracorporeal blood circulation.

According to one refinement of this above-mentioned aspect, the control unit is configured to execute the evaluation of sensor signals to detect a blood clot only when the actuator forms a constriction.

According to one refinement in turn of the above-mentioned aspect, the control unit is moreover configured to detect a quantitative parameter relating to the size of the constriction. Examples of such parameters are the distance between two clamping jaws, for example, of a tube clamp, the present cross section of a liquid conductor in the constriction, or also parameters relating to the actuator. Parameters relating to the actuator can also be those parameters which are not directly explicitly a parameter of the size of the constriction itself, but are unambiguously related to the size of the constriction and thus permit the conclusion of the size of the constriction. Such parameters can be the position of a positioning motor, the clamp position, the position of a linear motor, or the angular position, for example, of a rotatably mounted actuator magnet.

The further parameters of the extracorporeal blood circulation, on the basis of which the optimum width of the constriction is determined, are, for example, the set flow rate of the blood, the chronological progress of the treatment, the wall thickness of the tube/the blood tube/the blood line, the position of a roller of a tube roller pump (the latter, for example, to take into consideration the pulsation of the pressure in roller pumps in the detection of blood clots), viscosity or hematocrit of the blood. A further parameter can be the pressure in the blood tube when the constriction is still open. A parameter can be a value which is formed from the pressure in the tube and the volume flow rate in the tube. In addition to the parameter of pressure, a pressure increase upon the creation of the adjustable constriction with known blood volume flow can be a further parameter. In this way, for example, the influence of a patient connector such as a cannula on the pressure measurement can advantageously be compensated for.

In one advantageous refinement, the device according to the invention is used together with a pump which can generate a chronologically constant blood flow—for example, an impeller pump. Such pumps generate a uniform blood flow, whereas, e.g., roller pumps, membrane pumps, and syringe pumps generate oscillating blood flows. A further increased sensitivity of the device in the detection according to the invention of blood clots is particularly advantageously enabled by a chronologically constant blood flow. This is because if the underlying undisturbed blood flow is constant, deviations therefrom may be determined particularly easily.

In many medical devices, tube clamps are used, using which the flow through a tube, which has a flexible wall, can be clamped off. These clamps are present in many medical areas of application and are often constructed so that a tube is inserted into an open clamp. These clamps are generally used as a safety device, in particular for blood-conducting tubes. They are to ensure that, for example, the blood flow can be interrupted when, for example, an emergency occurs. For this purpose, the clamps have to have an open and a closed position and have to be switchable between the two operating states. For this purpose, such clamps often have means for clamping, a means for mechanical resetting (such as a spring), and an electrically controlled holding element. The resetting means provides force in order to close the clamp in the deenergized state of the holding element, by which the blood tube is clamped off and a blood line is interrupted. In some areas of medicine or medical technology, such tube clamps are prescribed to ensure patient safety.

Although a continuously pumping pump such as an impeller pump, a gear wheel pump, or a combination made up of multiple syringe pumps would be advantageous, use with cyclically operating pumps such as roller pumps or syringe pumps is also conceivable. For a device or a method using a cyclically operating pump for conveying the liquid in the extracorporeal blood circulation, a configuration is advantageous having a pressure transducer, which is arranged in order to determine the current pressure course over time in the liquid conveyed by the pump in a current conveyance cycle of the pump, to store a reference pressure course in a memory, wherein the memory can be associated with a device according to the invention or with a higher-order blood treatment device. The device according to the invention or possibly a higher-order blood treatment device then have an evaluation unit which determines pressure deviations in that a pressure course from the current conveyance cycle of the pump compares with the reference pressure course and detects pressure deviations by deviations. The reference pressure course can be obtained by acquiring the pressure course over time, for example, from one or more conveyance cycles preceding the current conveyance cycle of the pump, or also from historic data or mean values. For higher accuracy, the reference pressure course is preferably based on one or more conveyance cycles of the same pump which have taken place immediately or at least only a few conveyance cycles of the pump earlier than the current conveyance cycle.

According to one refinement of the present invention, a device according to the invention is implemented so that a tube clamp prescribed for patient safety is expanded in such a way that it has an actuator which can create an adjustable constriction. The tube clamp is thus expanded in such a way that in addition to the two typical operating states “completely open”, which is assumed during a proper blood treatment, and “completely closed”, which is assumed, for example, in emergency situations such as in the event of power failure, it can assume a third operating state “adjusted constriction for detecting blood clots”, which is neither completely open nor completely closed. This particularly advantageously enables the integration of the device for detecting blood clots in a device present in any case in many areas of application, such as the blood tube clamp. This enables a particularly compact, resource-preserving, and reliable integration.

According to one refinement of the above-described refinement having integration of the device according to the invention, the tube clamp is moreover refined in relation to known tube clamps such that it has two disk-shaped permanent magnets having more than two magnetic poles, which were generated by magnetization perpendicular to the surface extension of the disk, for example, using an electromagnetic coil arrangement in the manner of a hard drive write head (so-called polymagnets®). The implementation of tube clamps having magnets that can be polymagnetized particularly advantageously enables an efficient, compact, energy-saving device. This is because the various operating states can be configured by the adjustability of the magnetic regions such that only little energy is required to remain in the respective operating state.

In one particularly advantageous refinement of a system according to the invention, such an above-described tube clamp is used in a system together with a blood pump having continuous flow, in particular an impeller pump. A further increased sensitivity of the device in the detection according to the invention of blood clots is thus particularly advantageously enabled. A corresponding advantage is also obtained if pumps or combinations of pumps and/or further elements such as valves and throttles or junctions are interconnected so that a continuous blood flow results. Furthermore, a corresponding advantage also applies if blood is transported in an extracorporeal blood circulation by a pump which is not arranged in the blood circulation itself but conveys continuously-such as a gear wheel pump—and a medium conveying the pump propulsion—such as dialyzate.

Adjustable constriction in the context of this invention means that the constriction is not present fixedly and invariably in the blood circulation, but can be adjusted by means of an actuator. In the simplest case, an adjustable constriction means that an actuator of the device can create and remove the constriction or that an adjustable constriction can be created or removed again on a blood tube set by applying, for example, a mechanical means for constricting the cross section of the blood tube. In other words, the adjustable constriction simply does not mean a constriction which is present unchanged over the usage time (comprising treatment as well as preparation and follow-up of the treatment) on an extracorporeal blood circulation. It thus simply does not mean a constriction as is used by a cannula for the vessel access for connecting the extracorporeal blood circulation to a patient, for example. Such cannulas generally have a smaller line cross section than a blood tube, which typically makes up a significant part of an extracorporeal blood circulation. Other elements arranged fixedly and unchangeably on an extracorporeal blood circulation are also not meant, such as Luer connectors or chronologically unchanging constrictions in the flow cross section as are present in cassette elements or at transitions of various line sections, for example, because of the design. If the actuator simply does not create the constriction, the point of the blood circulation in which the actuator can create a constriction is not constricted by the actuator. In other words, in the simplest case the constriction can be added by means of the actuator or also cannot exist. It is thus ensured that a constriction is only created when a blood clot detection is actively being carried out. If no blood clot detection takes place, the cross section of the blood circulation is not reduced by the actuator to the constriction required for the measurement. The width of the constriction can preferably be predetermined here and matched to the detection according to the invention of blood clots.

An arrangement for detecting blood clots in the context of this application can comprise the following subgroups:
An extracorporeal blood circulation having an adjustable constriction on a tube section,
A device for detecting blood clots which a sensor that detects at least one parameter related to a property of the liquid in the extracorporeal blood circulation, and wherein there is an open fluidic connection between the tube section of the adjustable constriction and the tube section of the sensor, so that the sensor can detect a change of the measured parameter originating from a passage of a clot through the adjustable constriction.

Expressed in reverse: If there were a complete occlusion on the extracorporeal blood circulation between sensor and adjustable constriction, this would not be in accordance with the teaching of the present application because then the change of the measured parameter which is caused by the passage of a clot through the adjustable constriction could not be detected by the sensor.

The subgroups or components from the preceding paragraph can be sold as separate products here. A device for detecting a blood clot according to the invention can be sold separately and designed as a retrofitting set for extracorporeal blood circulation devices or can be sold as parts of such devices. Such devices can in particular be extracorporeal blood treatment devices. The production of an adjustable constriction for the detection of blood clots can be carried out by means of an actuator of the device—for example, an electromechanical tube clamp or a linear actuator. The blood tube set to be used in these embodiments can be here a blood tube set typical for the specific application, for example, in dialysis, therapeutic apheresis, or in cardiopulmonary support. The actuator of the device forms the constriction on a typical blood tube set. Alternatively, the production of an adjustable constriction for the detection of blood clots can be carried out by applying a manual clamp. The clamp can be sold individually or as part of a blood tube set for a treatment. In contrast to widespread manual blood tube clamps, the present clamp has to be of a nature such that it produces the adjustable constriction on the blood tube for the detection of blood clots as a partially open constriction, i.e., so that a blood circulation is not stopped but can continue. Such a clamp can be implemented as part of a blood tube set for single use or as a separate product.

According to one preferred embodiment of the device or the method according to the invention, the adjustable constriction and a pressure sensor can be/become arranged in the vicinity of one another, i.e., at most a few centimeters apart, along the course of a blood tube. More direct measurement for the detection of blood clots particularly advantageously results in this way, which is in addition less susceptible to disturbance than at a greater distance.

According to one preferred embodiment, the device and the method according to the invention are adapted so that the constriction can be executed on a blood tube for an extracorporeal blood treatment having an internal diameter between 3 mm and 15 mm in the relaxed state. The free cross-sectional area in the adjustable constriction preferably corresponds here to 1% to 99% of the cross-sectional area in the relaxed state. Furthermore, the area preferably corresponds to 10% to 90% of the cross-sectional area in the relaxed state. In any case, the width of the adjustable constriction is selected so that it enables a detection of a clot, preferably an improved detection.

According to one aspect of the invention, the width of the adjustable constriction which is created by the actuator of the present device on the blood circulation is predetermined and matched to the present detection of blood clots.

According to a further aspect of the method according to the invention for monitoring an extracorporeal blood circulation for detecting a blood clot in the extracorporeal blood circulation, the method steps are used of producing a constriction on the blood circulation using an actuator, measuring at least one parameter related to a property of the liquid in the line section having the constriction, using a sensor, evaluating the measurement data of the sensor to detect the passage of a blood clot through the constriction during a reinfusion method after a hemodialysis treatment, in which the patient is returned his blood simultaneously via two patient connectors, for example, both via an arterial and via a venous cannula, during the reinfusion. In this case, for example, no clot catcher can be arranged along the blood line in one of the two branches.

In one embodiment, the method is expanded by a sequence of method steps for individual adaptation of the width of the adjustable constriction as follows:
A defined blood volume flow is set
The adjustable constriction is created
The pressure and/or other parameters of the blood at the sensor change due to creation of the constriction
Adapting the width of the constriction until a predetermined target value of the pressure and/or other parameters is reached
Detecting clots

A control unit of a device having an actuator for creating a variable constriction can advantageously be configured analogously so as to adapt the width of the constriction.

It is thus advantageously possible to reach the optimum measurement range of a parameter for detecting blood clots-such as pressure or flow and/or further parameters.

A blood volume flow rate sensor can also be designated in short as a flow meter or flow sensor or volume flow sensor or volume flow rate sensor.

Blood tube and blood line can be used synonymously. Blood tubing set and blood tube set can be used synonymously. A blood tube set has at least one blood tube and can optionally have junctions. A section of a blood tube can be synonymous with a segment of a blood tube. An adjustable constriction according to the teaching of the present application is located on a section of a blood tube through which blood flows in the circulation during operation of an extracorporeal blood circulation. A segment of a blood tube can also have special properties such as material properties, material quality, tube cross section.

One aspect of the present invention relates to a system for use with a device or a method for detecting a blood clot at a constriction in an extracorporeal blood circulation comprising a blood tube or a blood line or a blood tube set configured for producing an extracorporeal blood circulation and a mechanical means for producing an adjustable constriction on a line section of the blood tube or the blood line or the blood tube set. The blood tube or the blood line or the blood tube set are configured for the production of an extracorporeal blood circulation and for connection to a human. For this purpose, the blood tube or the blood line or the blood tube set can have connecting means. The blood tube or the blood line or the blood tube set can be designed for single use. The blood tube or the blood line or the blood tube set can be of a nature such that a production of an extracorporeal blood circulation using an extracorporeal blood treatment device is enabled.

One preferred embodiment of the present invention relates to a manual mechanical tube clamp for producing an adjustable constriction on a section of a blood tube of an extracorporeal blood circulation for detecting blood clots in the extracorporeal blood circulation, wherein the clamp is of a nature such that it can assume and maintain a partially open position, wherein the clamp can partially compress a section of a blood tube in the partially open position, by which, on the one hand, a constriction is formed on the blood tube and due to which, on the other hand, the blood tube still remains permeable to a liquid at the constriction. Known clamps do not enable a partially open position as is required for detecting blood clots, however. One advantage of a manual mechanical tube clamp for producing an adjustable constriction for detecting blood clots is that the detection of blood clots without actuators of a treatment machine is enabled and a conventional blood tube set can also be used. The hygiene required in the medical area can be achieved in that, for example, the clamp consists of plastic and is intended for single use, is sterilized and packaged in a sterile manner or in that, for example, the clamp can be autoclaved and is manufactured from a medical steel. “Assume position” means that the clamp remains in the partially open state, thus is locked, until it is released therefrom again. A defined state is thus meant, a clamp position which the clamp can assume. This does not mean, for example, that commercially available household scissors can also not only be entirely opened and entirely closed and partial opening exists in between: This would not correspond to a defined state, a specific clamped position, as is meant here.

One aspect of the present invention relates to a blood tube set for extracorporeal blood treatment for the detection of blood clots in an extracorporeal blood circulation comprising a manual mechanical tube clamp as was described in the preceding paragraph, and a blood tube which has an arterial end for the connection to a patient access. It is thus particularly advantageously ensured that tube and clamp are matched to one another so that an adjustable constriction in the meaning of the present teaching can be formed using the clamp on the tube.

According to one refinement of the blood tube set described in the preceding paragraph for detecting blood clots in an extracorporeal blood circulation, which comprises a manual mechanical tube clamp which can assume a partially open position, the blood tube set additionally comprises at least one mechanical manual tube clamp which completely blocks the tube in a clamped position, wherein this additional tube clamp does not have a partially open position, however. This means a typical manual tube clamp, using which a blood tube can be completely blocked by pressing off. The blood tube set thus advantageously comprises at least two different clamps for the different tasks. Embodiments of this refinement can also comprise, however, two, three, or more typical manual tube clamps which completely block the tube position in a clamping position. In one refinement, the clamps are equipped with an identifier so that risk of confusion is reduced.

According to one refinement of a blood tube set for detecting blood clots in an extracorporeal blood circulation, which comprises a manual mechanical tube clamp that can assume a partially open position, as is described in one of the two preceding paragraphs once with and once without a further additional mechanical clamp, which can completely interrupt the blood flow in that it completely clamps off the tube in a clamping position, the blood tube set is of a nature such that using the clamp, which can assume a partially open position, the adjustable constriction can be formed on a section of the blood line in one of the following areas by arranging the clamp and by transferring the clamp into the partially open state:
[Alternative AA] between the arterial end of the blood line, thus the access to the patient (H), and a pump tube segment, which is a section of a blood tube insertable into a blood pump, wherein the blood tube set comprises the pump tube segment; or
[Alternative BB] between a manual mechanical tube clamp which does not have a partially open position, but completely presses off the blood tube in its clamping position, and a pump tube segment, which is a section of a blood tube insertable into a blood pump, wherein the blood tube set comprises the pump tube segment; or
[Alternative CC] between a manual mechanical tube clamp which does not have a partially open position, but completely presses off the blood tube in its clamping position, and a device for an arterial pressure measurement of the blood tube set, wherein the blood tube set comprises the device for an arterial pressure measurement; or
[Alternative DD] between a device for an arterial pressure measurement of the blood tube set and a pump tube segment which is a section of a blood tube insertable into a blood pump, wherein the blood tube set comprises the pump tube segment, wherein—if there are multiple blood pumps and/or pump tube segments—that pump tube segment is meant which is closest to the arterial end of the blood line, wherein the blood tube set comprises the device for an arterial pressure measurement and the pump tube segment;
or
[Alternative EE] between a fluidic interface configured for giving an infusion and a pump tube segment of the blood tube set which is a section of a blood tube insertable into a blood pump, wherein the blood tube set comprises the interface for giving an infusion and a blood pump and the pump tube segment; or
[Alternative FF] is arranged upstream of the access of the patient (H) at least 30 cm upstream of the arterial end of the blood line, thus the access of the patient (H); or
[Alternative GG] at least at the distance x upstream of the arterial end of the blood line, thus the access of the patient (H), according to the following formula for determining the distance x or

$$x ( Q , D , T ) = \frac{Q [ \frac{{cm}^{3}}{s} ] * T [ s ]}{π * {( \frac{D}{2} )}^{2} [ {cm}^{2} ]} ;$$

or
[Alternative HH] at least at the distance x upstream of the arterial end of the blood line, thus the access of the patient (H), according to the following formula for calculating the distance x.

$$x = 30 cm * \frac{Q}{200 [ \frac{ml}{min} ]} * \frac{4.5 [ mm ]}{D}$$

It is particularly advantageous in alternatives AA to HH of this refinement that blood tube set and clamp are designed so that the arrangement of the adjustable constriction is enabled for locations favorable for detecting blood clots. Alternatives AA to HH are of a nature here such that they are not mutually exclusive: Depending on the parameter values and configuration of blood tube set, etc. it is thus possible that the calculations according to alternatives GG and HH supply the same value or a value corresponding to alternative FF. Two or three of these alternatives could thus already be fulfilled simultaneously. Furthermore, one of the alternatives AA to EE could simultaneously be fulfilled for a given tubing set, so that one or more of alternatives FF to HH and at least one of alternatives AA to EE can be fulfilled simultaneously. With known tubing sets, the range of alternative AA can comprise the ranges of alternatives BB to EE, the range of alternative BB can comprise the range of alternative CC and alternative EE. The range of alternative CC can comprise the range of alternative EE. Known tubing sets can be of a nature such that the range of alternative DD and the range of alternative CC are not provided in an overlapping manner. Known tubing sets can be of a nature such that the range of alternative DD and the range of alternative EE overlap. The term tube set/tubing set can be used synonymously for the term blood tube set/blood tubing set. For alternatives EE to HH, it can be particularly advantageous that due to the distance of the adjustable constriction from the patient access, sufficient reaction time is ensured for the system so that it is possible to prevent a detected blood clot from being transported to the patient.

One aspect of the present invention relates to an extracorporeal blood treatment device comprising a device according to the invention for detecting a blood clot, an electromagnetic safety tube clamp for pressing off a blood tube in the event of an imminent risk and/or a means for outputting an alarm and/or a blood pump, wherein the control unit of the blood treatment device is configured to close the safety tube clamp and/or output an alarm and/or stop the blood pump if the device for detecting blood clots detects a blood clot. In other words, according to this aspect an extracorporeal blood treatment device according to the invention is equipped with a device according to the invention for detecting a blood clot and in addition the extracorporeal blood treatment device comprises one or more of the active components of this list: electromagnetic safety tube clamp, means for outputting an alarm, blood pump. The statements at another point in this document apply, where an extracorporeal blood treatment device according to the invention having active components such as tube clamp, means for outputting an alarm, and blood pump is discussed. Since it is an active component of a device here, “tube clamp” does not mean a manual mechanical tube clamp but rather an electrical or electromagnetic or otherwise actuated tube clamp.

One aspect of the invention relates to a system for extracorporeal blood treatment of a patient comprising an extracorporeal blood treatment device having a device according to the invention for detecting blood clots in an extracorporeal blood circulation. The extracorporeal blood treatment device additionally comprises an electromagnetic safety tube clamp for pressing off a blood tube in the event of imminent risk and/or a means for outputting an alarm and/or a blood pump, wherein the control unit of the blood treatment device is configured to close the safety tube clamp and/or output an alarm and/or stop the blood pump if the device for detecting blood clots detects a blood clot. An extracorporeal blood treatment device generally has a control unit. It is possible that the device for detecting blood clots and the extracorporeal blood treatment device have separate control units. However, it is also possible that both functionalities are combined in one control unit. The control unit of the extracorporeal blood treatment device would then also have the functionality of the control unit of the device for detecting blood clots. Furthermore, the system comprises a blood tube set. In this embodiment of the invention, the device for detecting blood clots is arranged on a section of the blood line which is at a distance of at least 30 cm upstream of the access of the patient or which is calculated according to one of the two of the following alternative formulas for determining the distance x:

$$x ( Q , D , T ) = \frac{Q [ \frac{{cm}^{3}}{s} ] * T [ s ]}{π * {( \frac{D}{2} )}^{2} [ {cm}^{2} ]} or$$

$$x = 30 cm * \frac{Q}{200 [ \frac{ml}{min} ]} * \frac{4.5 [ mm ]}{D}$$

This advantageously enables blood clots to be detected by the device at a significant distance from the patient. The embodiments described elsewhere in this document for arranging the adjustable constriction at a specific distance, if not contradictory, also apply to this embodiment.

One aspect of the invention relates to a system for extracorporeal blood treatment of a patient comprising an extracorporeal blood treatment device having a device according to the invention for detecting blood clots in an extracorporeal blood circulation. The extracorporeal blood treatment device additionally comprises an electromagnetic safety tube clamp for pressing off a blood tube in the event of imminent risk and/or a means for outputting an alarm and/or a blood pump, wherein the control unit of the blood treatment device is configured to close the safety tube clamp and/or output an alarm and/or stop the blood pump if the device for detecting blood clots detects a blood clot. An extracorporeal blood treatment device generally has a control unit. It is possible that the device for detecting blood clots and the extracorporeal blood treatment device have separate control units. However, it is also possible that both functionalities are combined in one control unit. The control unit of the extracorporeal blood treatment device would then also have the functionality of the control unit of the device for detecting blood clots. Furthermore, the system comprises a blood tube set. In the system according to this embodiment, the blood tube set and the blood treatment device are arranged in relation to one another so that an adjustable constriction for detecting blood clots is formed on a section of the blood line in one of the following areas by means of an actuator or by means of a mechanical clamp:
[Alternative 21AA] between the arterial end of the blood line, thus the access to the patient, and a pump tube segment, which is a section of a blood tube insertable into a blood pump, wherein the blood tube set comprises the pump tube segment;
[Alternative 21BB] between a manual mechanical tube clamp which does not have a partially open position, but completely presses off the blood tube in its clamping position, and a pump tube segment, which is a section of a blood tube insertable into a blood pump, wherein the blood tube set comprises the pump tube segment;
[Alternative 21CC] between a manual mechanical tube clamp which does not have a partially open position, but completely presses off the blood tube in its clamping position, and a device for an arterial pressure measurement of the blood tube set, wherein the blood tube set comprises the device for an arterial pressure measurement;
[Alternative 21DD] between a device for an arterial pressure measurement of the blood tube set and a pump tube segment which is a section of a blood tube insertable into a blood pump, wherein the blood tube set comprises the pump tube segment, wherein—if there are multiple blood pumps and/or pump tube segments—that pump tube segment is meant which is closest to the arterial end of the blood line, wherein the blood tube set comprises the device for an arterial pressure measurement and the pump tube segment;
[Alternative 21EE] between a fluidic interface configured for giving an infusion and a pump tube segment of the blood tube set which is a section of a blood tube insertable into a blood pump, wherein the blood tube set comprises the interface for giving an infusion and a blood pump and the pump tube segment.

The statements made above with respect to alternatives AA to EE on the locations of the adjustable constriction including the advantages apply to alternatives 21AA to 21EE.

When reference is made in the present document to “pressing off a tube” (entirely or partially), this means “blocking off”. I.e., it is not central whether the adjustable constriction is created by pressing off or the cross section is reduced in another way. Therefore “pressing off” can also be interpreted as “blocking off”. This is also to be understood for the partially open position of an adjustable constriction.

One embodiment of the method according to the invention for monitoring an extracorporeal blood circulation for detecting a blood clot in the extracorporeal blood circulation comprises at least the following steps:
Producing an adjustable constriction on the blood circulation by compressing a section of a blood tube, wherein the adjustable constriction has a width such that the blood can continue to flow through the constriction, using an actuator or by setting or applying a manual mechanical clamp, wherein the manual clamp assumes a partially open position,
Measuring at least one parameter related to a property of the liquid in the line section having the constriction using a sensor,
Evaluating the measurement data of the sensor to detect the passage of a blood clot through the constriction using a control unit.

The statements made above on methods according to the invention, including the advantages, apply to this embodiment of the method.

### DESCRIPTION OF THE FIGURES

In the figures:

FIG. 1 shows a diagram of an extracorporeal blood circulation, which is connected to the blood circulation of a human, having a device according to the invention;

FIG. 2 shows two graphs of the courses of liquid pressure and volume flow speed on the same time axis;

FIG. 3 shows a diagram of an extracorporeal blood circulation, which is configured for a hemodialysis treatment, having a device according to the invention;

FIG. 4 shows a modification of the diagram of an extracorporeal blood circulation, which is connectable to the blood circulation of a human, having a device according to the invention like FIG. 1 but with the pump at a different position.

FIG. 1 schematically shows an extracorporeal blood circulation 300 having a device 100 presented here for detecting blood clots in an extracorporeal blood circulation. In the schematic representation, a human His indicated on the left side, to whom the extracorporeal blood circulation is connected in the example shown. The upper branch of the blood circulation 300 leads away from the human H via a blood pump 10 to an adjustable constriction 20 of the blood line. The actuator D of the device can create the adjustable constriction 20 in that it reduces the cross section of the blood line in a section 25. Downstream of the constriction 20, a pressure sensor P for measuring the liquid pressure in the extracorporeal blood circulation 300 and a volume flow speed sensor Q for measuring the flow speed of the extracorporeal blood circulation 300 are arranged. In a fundamental variant, however, only one of these two sensors P, Q is required. The second sensor is optional. After passing the sensors, the blood flows via the lower branch of the extracorporeal blood circulation 300 back to the human H. An elastic blood tube is typically at the location of the constriction 20. An actuator D can itself create a constriction or, as shown here, can be connected to a further element E which compresses the section of the blood tube and thus forms the constriction. The constriction 20 is adjustable by means of the actuator D. An exemplary structure is shown in which a constriction 20 is formed and the blood tube is accordingly deformed on a section 25 so that the cross section is reduced. The control unit CU of the device 100 presented here is connected to the actuator D of the adjustable constriction and the sensor or sensors P, Q. The sequence shown here in the flow direction of pump 10, constriction 20, and sensor or sensors P, Q is only by way of example. Arrangements in which the sensor or sensors P, Q are arranged between the pump 10 and the constriction 20, or other arrangements, are also conceivable. In the embodiment shown, it can happen that a blood clot which is transported starting from the blood pump P further through the extracorporeal blood circulation 300 then reaches the constriction 20. The presence of a blood clot in the constriction 20 will result in a change of the flow Q and the pressure P. The control unit CU acquires the measured values of the sensor or sensors and can thus detect a blood clot in the constriction.

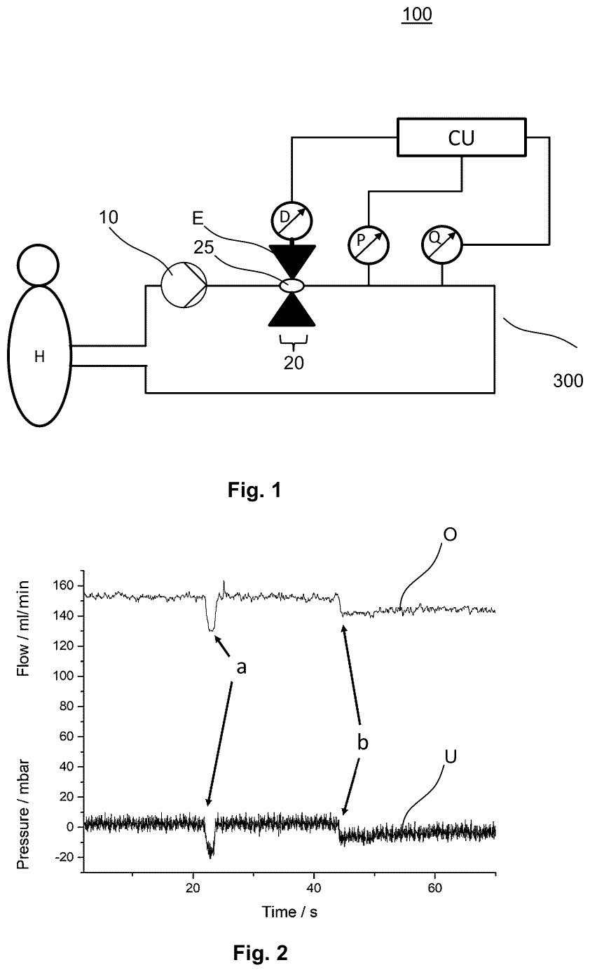

FIG. 2 shows a diagram in which measurement data recorded using a device 100 according to the invention in the arrangement from FIG. 1 are plotted. Two separate graphs O, U are shown in a diagram. The upper graph O shows measurement data of a volume flow rate measured using a volume flow sensor Q and the lower graph U shows measurement data of a pressure measurement measured using a pressure sensor P. The measurement data were recorded simultaneously on the same device 100 and share a common abscissa axis, on which the time “time” is plotted in seconds s. The two graphs have different value ranges and units on the ordinate axis: The upper graph O shows the volume flow rate “flow” in ml/min, wherein many values are in the range of approximately 140-150 ml/min. The lower graph U shows the pressure relative to a reference pressure in mbar, wherein the values are initially in the single-digit positive range and then also sink to almost approximately-20 mbar. Although the variables which are plotted in the two different graphs in the ordinate differ, it is expedient to plot them on a shared axis. The arrows on the left in the diagram, approximately at “Time”=22 s on the abscissa axis, mark a short-term sinking a of flow and pressure which was caused by the passage of a blood clot through the constriction 20. The short-term sinking of flow and/or pressure is an example of a signature in the measurement signals, on the basis of which the device 100 according to the invention detects a passage of a blood clot through the adjustable constriction 20. On the right in the diagram, approximately at “Time”=44 s, arrows indicate a further abrupt sinking b of flow and pressure, wherein flow and pressure remain at the low level after the sinking b, however. This is the signature of a clot which has stuck in the constriction 20 of an arrangement having a continuously pumping pump such as an impeller pump. In the case of a different order of the elements pump 10, constriction 20, and pressure meter P and/or volume flow rate sensor Q, a passage of a clot through the constriction 20 can be accompanied by the increase of pressure and flow. Depending on the arrangement, however, a flow value can also sink while a pressure rises.

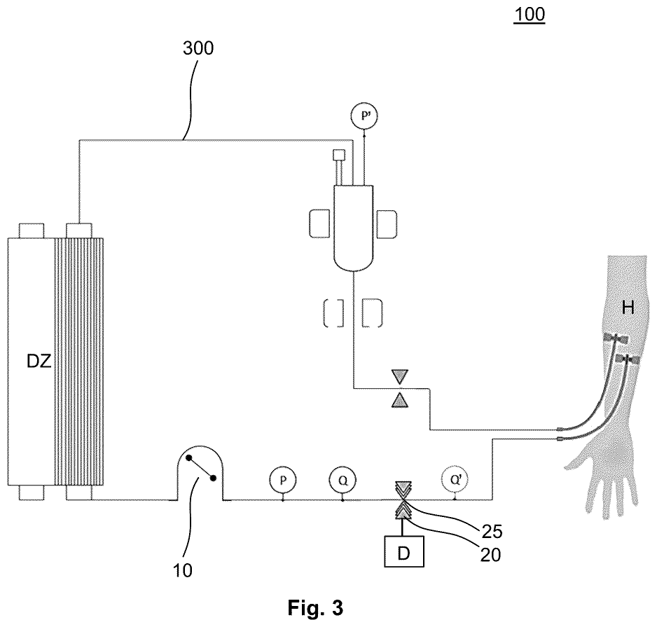

FIG. 3 shows the device 100 according to the invention in an exemplary embodiment which is configured for a hemodialysis treatment using a dialyzer D in the extracorporeal blood circulation 300. An arm of a patient H having two connectors, shown as an arterial and a venous blood cannula, is indicated on the right side of the image. During a dialysis treatment, in the lower branch, blood flows from the patient H to a dialyzer DZ and initially passes here an optional first flow meter Q′, then the actuator D, which can create an adjustable constriction 20 on a line section 25 of the blood line, then a second flow meter Q and a pressure meter P as well as a blood pump 10, optionally symbolized here as a roller pump. Instead of a roller pump P, other blood pumps P such as impeller pumps P can also be used. During the phase of reinfusion, the blood present in the extracorporeal blood circulation is reinfused in the patient. In so-called close-loop reinfusion, in the arrangement from FIG. 3, blood is transported in the lower branch from the dialyzer DZ via the blood pump 10 and the constriction 20 back to the patient H. A detection of blood clots according to the invention is already possible if only one sensor, thus a blood volume flow rate sensor Q or a pressure meter P, are arranged in this branch of the extracorporeal blood circulation. The further sensors are optional. However, the arrangement of both pressure sensor P and flow sensor Q and the evaluation thereof in consideration together is advantageous insofar as a higher sensitivity can be achieved. In the arrangement of FIG. 3, the passage of blood clots, depending on which of the partially optional sensors are arranged, would have a different sign than shown in FIG. 2: In an exemplary embodiment according to FIG. 3 having only one pressure sensor P and one volume flow rate sensor Q between the adjustable constriction 20 and the blood pump, resulting signatures of blood clots were measured as described hereinafter. In treatment operation, a blood clot in the constriction 20 would result in a reduction of pressure and flow in the sensors. In the case of a reinfusion, a clot in the constriction 20 would result in a rise of the measured pressure and in a drop of the measured volume flow rate.

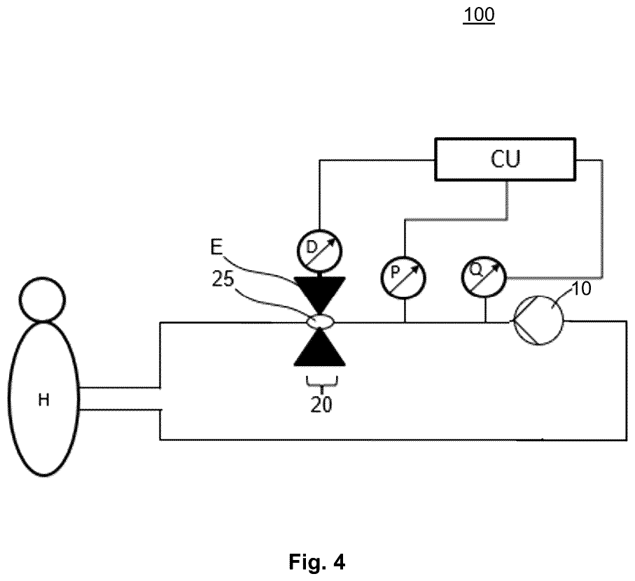

FIG. 4 schematically shows the extracorporeal blood circulation 300 having a device 100 presented here for detecting blood clots in an extracorporeal blood circulation as in FIG. 1, only with the difference that the pump is arranged upstream of sensors and constriction. A representation analogous to the data plot from FIG. 2 would have different signs in this case upon the presence of blood clots. The arrangement shown here has the pressure sensor arranged before the constriction in the flow direction. A high level of sensitivity of the pressure measurement for clots in the constriction is thus particularly advantageously enabled.

1. A device for detecting blood clots in an extracorporeal blood circulation, connectable to an extracorporeal blood circulation having an adjustable constriction, comprising:
at least one sensor, which acquires at least one parameter related to a property of the liquid in the extracorporeal blood circulation,
a control unit, which is configured so that a measurement signal from the at least one sensor is evaluated, and that on the basis of this evaluation of the parameter measured by the sensor, the passage of a blood clot through the constriction and/or sticking of a blood clot in the constriction is capable of being detected,
wherein the device is configured such that the device is capable of detecting blood clots at an adjustable constriction which has been adjusted by constricting a blood tube using an actuator or a mechanical clamp.

2. The device of claim 1, wherein the sensor is a flow sensor, which measures the current flow rate of the liquid, or a pressure sensor, which measures the current pressure of the liquid.

3. The device of claim 1, having at least one flow sensor and at least one pressure sensor, wherein the control unit is configured so that it uses the measured values of at least one flow sensor and at least one pressure sensor in consideration together to detect blood clots.

4. The device of claim 1, comprising an actuator, which is capable of creating an adjustable constriction on the extracorporeal blood circulation, in that it reduces the cross section of a section of a blood line.

5. The device of claim 4, which is designed as a tube clamp or as part of a tube clamp into which a medical tube is insertable.

6. The device of claim 5, wherein the actuator is capable of assuming at least three different positions, namely: (a) a closed position, in which the extracorporeal blood circulation does not have a free line cross section at one point, thus is blocked, (b) an open position, in which the extracorporeal blood circulation does not have a constriction at the actuator, and (c) a third, partially open position, which is between the closed and the open position, in which an adjusted constriction is capable of being created to detect blood clots on an inserted tube.

7. The device of claim 6, wherein the tube clamp has so-called polymagnets, which comprise a plurality of magnetized areas so that the clamp has at least three different positions.

8. The device of claim 5, wherein a spacer is arranged between clamp elements of the clamp to thus implement the partially open clamp position at the constriction, and wherein the device only optionally comprises an actuator that creates an adjustable constriction on the extracorporeal circulation.

9. The device of claim 4, wherein the actuator and the control unit are configured so that the device is capable of assuming at least two different operating states, in each of which the actuator forms the constriction, wherein the predetermined width of the constriction is different in the different operating states.

10. The device of claim 9, wherein the control unit is configured to select the width of the constriction according to a predetermined scheme and adjust it at the actuator to detect blood clots in consideration of a parameter of the extracorporeal blood circulation.

11. The device of claim 4, wherein the width of the constriction is variable and the control unit is configured to adjust the constriction for the operation for detecting blood clots in consideration of a parameter of the extracorporeal blood circulation so that the sensitivity and/or the robustness of the detection is optimized.

12. The device of claim 11, wherein the control unit is configured, during a medical treatment using the extracorporeal blood circulation, to calculate the optimal width of the constriction as a function of a parameter of the extracorporeal blood circulation and/or a parameter of the treatment and adjust it using the actuator.

13. A blood tube set configured for use with the device of claim 1, comprising a blood tube or blood line and a mechanical means for adjusting a constriction on a line section of the blood tube or the blood line.

14. A manual mechanical tube clamp for producing an adjustable constriction on a section of a blood tube of an extracorporeal blood circulation for detecting blood clots in the extracorporeal blood circulation, wherein the clamp is of a nature such that the clamp is capable of assuming and maintaining a partially open position, wherein the clamp is capable of partially compressing can partially compress a section of a blood tube in the partially open position, by which, on the one hand, a constriction is formed on the blood tube and due to which, on the other hand, the blood tube still remains permeable to a liquid at the constriction.

15. A blood tube set for extracorporeal blood treatment for detecting blood clots in an extracorporeal blood circulation comprising the manual mechanical tube clamp of claim 14 and a blood tube, which has an arterial end for the connection to a patient access.

16. The blood tube set for detecting blood clots in an extracorporeal blood circulation according to claim 15 additionally comprising a mechanical manual tube clamp, which completely blocks off the tube in a clamping position, but does not have a partially open position.

17. The blood tube set for detecting blood clots in an extracorporeal blood circulation according to claim 15, of a nature such that using the clamp, which is capable of assuming a partially open position, the adjustable constriction is capable of forming on a section of the blood line in one of the following areas by arranging the clamp and by transferring the clamp into the partially open position:
between the arterial end of the blood line, thus the access to the patient, and a pump tube segment, which is a section of a blood tube insertable into a blood pump, wherein the blood tube set comprises the pump tube segment; or
between a manual mechanical tube clamp which does not have a partially open position, but completely presses off the blood tube in its clamping position, and a pump tube segment, which is a section of a blood tube insertable into a blood pump, wherein the blood tube set comprises the pump tube segment; or
between a manual mechanical tube clamp which does not have a partially open position, but completely presses off the blood tube in its clamping position, and a device for an arterial pressure measurement of the blood tube set, wherein the blood tube set comprises the device for an arterial pressure measurement; or
between a device for an arterial pressure measurement of the blood tube set and a pump tube segment which is a section of a blood tube insertable into a blood pump, wherein the blood tube set comprises the pump tube segment, wherein—if there are multiple blood pumps and/or pump tube segments—that pump tube segment is meant which is closest to the arterial end of the blood line, wherein the blood tube set comprises the device for an arterial pressure measurement and the pump tube segment; or
between a fluidic interface configured for giving an infusion and a pump tube segment of the blood tube set which is a section of a blood tube insertable into a blood pump, wherein the blood tube set comprises the interface for giving an infusion and a blood pump and the pump tube segment; or
is arranged upstream of the access of the patient at least 30 cm upstream of the arterial end of the blood line, thus the access of the patient; or
at least at the distance x upstream of the arterial end of the blood line, thus the access of the patient, according to the following formula for determining the distance x
$$x ( Q , D , T ) = \frac{Q [ \frac{{cm}^{3}}{s} ] * T [ s ]}{π * {( \frac{D}{2} )}^{2} [ {cm}^{2} ]} ;$$
or
at least at the distance x upstream of the arterial end of the blood line, thus the access of the patient, according to the following formula for determining the distance x.
$$x = 30 cm * \frac{Q}{200 [ \frac{ml}{min} ]} * \frac{4.5 [ mm ]}{D} .$$

18. An extracorporeal blood treatment device comprising: the device of claim 1, an electromagnetic safety tube clamp for pressing off a blood tube in the event of imminent risk and/or a means for outputting an alarm and/or a blood pump, wherein the control unit of the blood treatment device is configured to close the safety tube clamp and/or output an alarm and/or stop the blood pump when the device for detecting blood clots detects a blood clot.

19. The extracorporeal blood treatment device of claim 18, wherein the blood treatment device is a hemodialysis machine.

20. A system for extracorporeal blood treatment of a patient comprising the extracorporeal blood treatment device of claim 18 and a blood tube, wherein the adjustable constriction for detecting blood clots is formed on a section of the blood line, so that the constriction is arranged at least 30 cm upstream of the access of the patient or so that the constriction is arranged upstream of the access of the patient at least at the distance x according to one of the two following alternative formulas for determining the distance x.
$$x ( Q , D , T ) = \frac{Q [ \frac{{cm}^{3}}{s} ] * T [ s ]}{π * {( \frac{D}{2} )}^{2} [ {cm}^{2} ]} or$$
$$x = 30 cm * \frac{Q}{200 [ \frac{ml}{min} ]} * \frac{4.5 [ mm ]}{D} .$$

21. A system for extracorporeal blood treatment of a patient comprising the extracorporeal blood treatment device of claim 18 and a blood tube set, wherein the blood tube set and the blood treatment device are arranged in relation to one another so that an adjustable constriction for detecting blood clots is formed on a section of the blood line in one of the following areas by means of an actuator or by means of a mechanical clamp:
between the arterial end of the blood line, thus the access to the patient, and a pump tube segment, which is a section of a blood tube insertable into a blood pump, wherein the blood tube set comprises the pump tube segment;
between a manual mechanical tube clamp which does not have a partially open position, but completely presses off the blood tube in its clamping position, and a pump tube segment, which is a section of a blood tube insertable into a blood pump, wherein the blood tube set comprises the pump tube segment;
between a manual mechanical tube clamp which does not have a partially open position, but completely presses off the blood tube in its clamping position, and a device for an arterial pressure measurement of the blood tube set, wherein the blood tube set comprises the device for an arterial pressure measurement;
between a device for an arterial pressure measurement of the blood tube set and a pump tube segment which is a section of a blood tube insertable into a blood pump, wherein the blood tube set comprises the pump tube segment, wherein—if there are multiple blood pumps and/or pump tube segments—that pump tube segment is meant which is closest to the arterial end of the blood line, wherein the blood tube set comprises the device for an arterial pressure measurement and the pump tube segment;
between a fluidic interface configured for giving an infusion and a pump tube segment of the blood tube set which is a section of a blood tube insertable into a blood pump, wherein the blood tube set comprises the interface for giving an infusion and a blood pump and the pump tube segment.

22. A method for monitoring an extracorporeal blood circulation for detecting a blood clot in the extracorporeal blood circulation comprising at least the steps:
producing an adjustable constriction on the blood circulation by compressing a section of a blood tube, wherein the adjustable constriction has a width such that the blood is capable of continuing to flow through the constriction, using an actuator or by setting or applying a manual mechanical clamp, wherein the manual clamp assumes a partially open position,
measuring at least one parameter related to a property of the liquid in the line section having the constriction using a sensor,
evaluating the measurement data of the sensor to detect the passage of a blood clot through the constriction using a control unit.

23. A method for controlling an extracorporeal blood treatment device comprising the method according to claim 22 and additionally the step:
closing a tube clamp and/or outputting an alarm and/or stopping a blood pump when the passage of a blood clot through the constriction is established.
